(19) United States
(12) Patent Application Publication
(10) Pub. No.: US 2009/0055987 A1
(43) Pub. Date: Mar. 5, 2009

(54) Airflow Headgear for a Welding Helmet

(75) Inventors: William Becker, Manitowoc, WI (US); Kenneth S. Dobson, Northville, MI (US); Kui-Chiu Kwok, Gurnee, IL (US); Eric Sommers, Appleton, WI (US)

Correspondence Address: FLETCHER YODER (ILLINOIS TOOL WORKS INC.), P.O. BOX 692289, HOUSTON, TX, 77269-2289, US

(73) Assignee: Illinois Tool Works Inc., Glenview, IL (US)

(21) Appl. No.: 12/201,185

(22) Filed: Aug. 29, 2008

Related U.S. Application Data
(60) Provisional application No. 60/967,511, filed on Sep. 5, 2007.

(51) Int. Cl. A61F 9/06; A42C 5/04; A42B 1/24

(52) U.S. Cl. 2/8.6; 2/171.3; 2/209.13

(57) ABSTRACT

An airflow system is provided for a welding helmet. The airflow system includes an air intake with a filter, a blower, and a battery mounted to the rear of headgear for a welding helmet. A manifold wraps around the side and front of the headgear and directs air from the blower to the front of the headgear. The manifold includes a lower vent for directing air onto a user's face and an upper vent for directing air over a user's head toward the rear of the headgear. The vents are intended to create positive pressure to impede the entry of unfiltered air into the user's breathing zone. The manifold further includes a portion of flexible tubing for adjusting the size of the airflow system in conjunction with adjustment of the headgear diameter. The airflow system generally conforms to the structure and shape of the headgear to maintain a location close to the user's head.

### CROSS REFERENCE TO RELATED APPLICATIONS

This application is a Non-Provisional Patent Application of U.S. Provisional Patent Application No. 60/967,511, entitled “Airflow Headgear for a Welding Helmet,” filed Sep. 5, 2007, which is herein incorporated by reference.

### BACKGROUND

The invention relates generally to airflow systems for welding helmets.

Welding can be a heat intensive process, especially during the summer months in outdoor locations or in plants without air conditioning. Further, welders typically wear equipment, such as leather gloves, long sleeve jackets, and welding helmets to cover the eyes, face, and neck. The equipment may add to the heat intensive aspects of the welding process and may reduce comfort by allowing air to stagnate, particularly around a welder's face. Further, the welding process may generate smoke, fumes, and various gases that add to the discomfort of welders.

Various arrangements have been devised in attempts to provide cooling and improved air quality to welders. However, past attempts often require separate equipment that may be expensive, inconvenient, uncomfortable, and/or heavy. For example, a welder may not take the time to attach additional equipment that is separate from the welder's current equipment. There is a need, therefore, for a portable and inexpensive airflow system that can be integrated into welding headgear.

### BRIEF DESCRIPTION

The present invention provides a novel approach to this problem designed to resolve certain of these drawbacks in the art. In particular, the invention provides an airflow system that may be integrated into or attached to headgear for a welding helmet. The airflow system includes an air intake, battery, and blower or fan located at the rear of the headgear. The rear location allows the air intake to be located away from the welding fumes and particles, and also allows the system to be mounted close to a user's head to improve balance. A manifold directs air from the blower around the side of a user's head to vents located on the front of the headgear. The vents include two sets of tubes: a bottom set for directing air down toward a user's face and a top set for directing air up and over a user's head. The combination of top and bottom tubes is intended to create positive pressure that impedes outside air from entering the user's breathing zone. Certain embodiments may also include side tubes for directing air over the sides of a user's face to create positive pressure. The airflow system also includes a flexible conduit integrated into the manifold to allow size adjustments. In certain embodiments, the flexible conduit may be expanded or contracted as the diameter of the headgear is adjusted using a knob.

### DRAWINGS

These and other features, aspects, and advantages of the present invention will become better understood when the following detailed description is read with reference to the accompanying drawings in which like characters represent like parts throughout the drawings, wherein:

FIG. 1 is an illustration of a welder wearing an exemplary welding helmet with an integrated airflow system in accordance with aspects of the present invention;

FIG. 2 is a perspective view of an exemplary headgear and airflow system that may be used with the welding helmet of FIG. 1;

FIG. 3 is a perspective view of the airflow system shown in FIG. 2 illustrating the airflow system detached from the headgear;

FIG. 4 is a detailed top perspective view of the airflow system illustrating attachment of the airflow system to the headgear;

FIG. 5 is a perspective view of an adapter bracket that may be used to attach the airflow system to the headgear;

FIG. 6 is a detailed perspective view of mounting structures for attaching the bracket of FIG. 5 to the airflow system;

FIG. 7 is a top perspective view of the airflow system illustrating the interior of the manifold;

FIG. 8 is a bottom perspective view of the manifold portion of the airflow system;

FIG. 9 is a bottom perspective view of the airflow system illustrating the airflow through the airflow system;

FIG. 10 is a rear perspective view of the airflow system illustrating the interior of the blower enclosure;

FIG. 11 is a rear perspective view of the airflow system illustrating the interior of the power supply enclosure;

FIG. 12 is a detailed perspective view of the power supply enclosure;

FIG. 13 is a perspective view of the airflow system illustrating the automatic on/off switch; and

FIG. 14 is an electrical schematic of exemplary circuitry for the airflow system;

### DETAILED DESCRIPTION

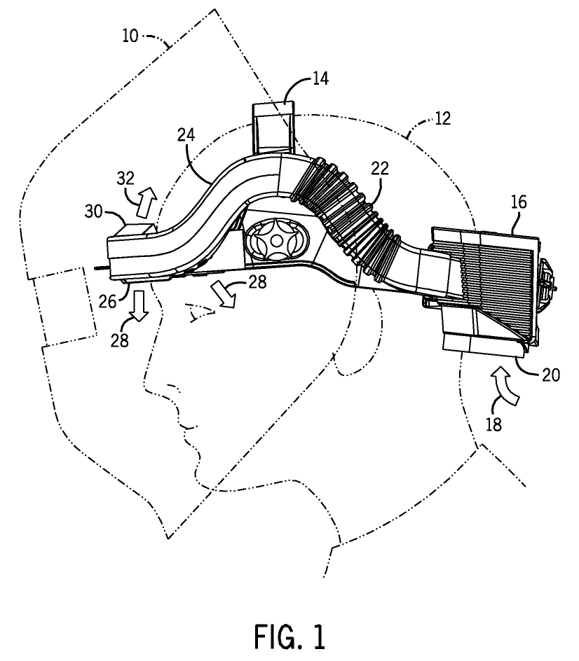

FIG. 1 illustrates an exemplary welding helmet 10 that incorporates an integrated airflow system in accordance with aspects of the invention. The welding helmet 10 may be constructed of a thermal plastic resin and may cover the face of the welder. The welding helmet 10 may be attached to a headgear 14 that is worn by the wearer or user 12. The headgear 14 generally includes straps that extend around the user's head and over the top of the head to provide support and stability for the welding helmet 10. An airflow system 16 may be attached to or integrated into the headgear 14 and may generally follow the circumference of the headgear 14. In certain embodiments, the airflow system may be permanently attached to the headgear. In such an embodiment, the headgear itself may serve as part of the air conduit. However, in other embodiments, the airflow system may be affixed to the headgear using mounting brackets or other attachment methods. By generally following the circumference of the headgear 14, the airflow system 16 may be located close to the user's head to promote stability and balance when worn. The close location to the user's head also is intended to provide airflow close to the user's face.

Air 18 may enter the airflow system through an air intake 20 that is located at the rear of the headgear 14. The rear location allows the air 18 to enter the airflow system 16 from an area behind the user 12. The area behind the user 12 may contain a lower amount of welding fumes, particulates, and gases than the area in front of the user 12 where the welding generally may occur. After entering the air intake 20, the air may flow through a flexible conduit 22 into a manifold 24. The flexible conduit 22 may include flexible tubing disposed on one side of the user's head, generally along one of the headgear straps. The manifold 24 may direct the air to a lower vent 26 where the air exits the airflow system 16 and is directed transversely toward a user's face and breathing zone, as indicated generally by the arrows 28. The air also may be directed transversely through an upper vent 30, as indicated generally by arrows 32. From upper vent 30, the air 32 may flow up and over a user's head generally toward the rear of the airflow system 16. The air from the vents 26 and 30 may function to provide positive pressure and impede air that has not flowed through the airflow system 16 from entering the user's breathing zone. In other embodiments, a fabric structure may be attached to the welding helmet to create a seal or barrier between the welding helmet 10 and the head and/or neck of the user 12 to impede unfiltered air from entering the user's breathing zone.

The headgear 14 includes knobs 34 located on opposite sides of the headgear for attaching the welding helmet 10 to the headgear 14. The knobs 34 may be rotated to adjust the angle of the helmet 10 with respect to the user's face. When the helmet 10 is attached to the headgear 14, the airflow system 16 may provide filtered air to the user's breathing zone, as generally indicated by the arrows 28. The filtered air may flow between the helmet 10 and the user's face.

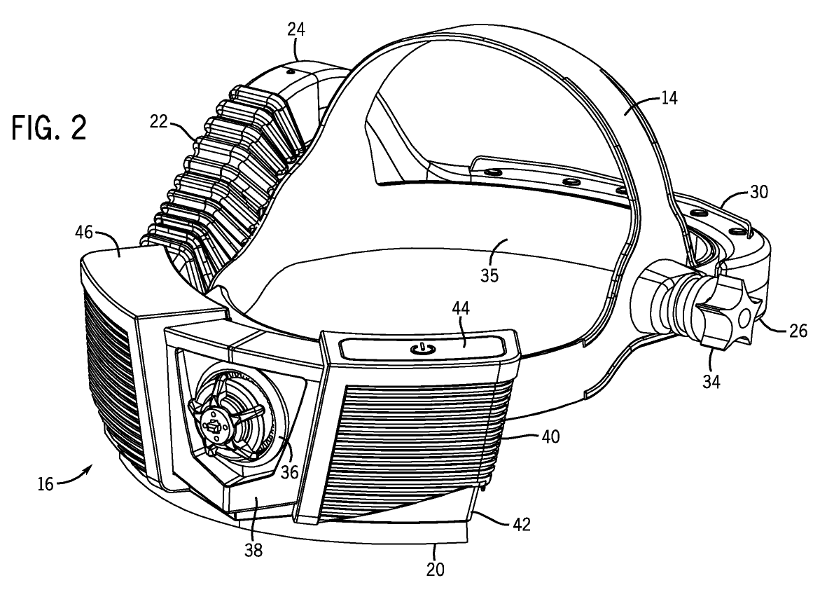

FIG. 2 illustrates the airflow system 16 attached to the headgear 14. The headgear 14 may be compatible with a variety of welding helmet brands and styles. The headgear 14 includes a lower strap 35 designed to encircle the head of a user. The airflow system 16 is generally disposed along the strap 35 and is intended to encircle a portion of the user's head and generally follow the circumference of the strap. The airflow system 16 includes an adjustment knob 36 that may be rotated to adjust the size of the circumferential size of the headgear 14. As the knob 36 is adjusted, the flexible conduit 22 may be expanded or contracted in conjunction with the adjustment of the strap 35. In other embodiments, the knob may be located on the side of the strap 35. The knob 36 may be sufficiently large in size to allow a user to rotate the knob while wearing gloves.

The knob 36 may be located within an enclosure 38 that houses the rear portion of the airflow system 16. The enclosure may be constructed of Nylon 6, 6, plastic or other suitable material and may provide support for the internal components and protect them from environmental contaminants, such as dust and debris. In certain embodiments, the enclosure 38 may have a chrome finish and smooth ribbed surfaces. The enclosure 38 is generally located on the rear of the headgear 14 in order to promote stability and balance when the headgear 14 is worn by a user.

The enclosure 38 includes a battery enclosure 40 that houses a power supply, such as a battery, and a circuit. The enclosure 40 includes a lower surface 42 and an on/off switch 44. The lower surface 42 may support a user's thumb while the user depresses the on/off switch 44 with his fingers. The on/off switch 44 may be sufficiently large in size to allow depression of the switch while a user is wearing gloves. In certain embodiments, the switch 44 may be actuated once to turn the system 16 on and then actuated a second time to turn the system off. The on/off switch may also be surrounded by a rubber gasket or other seal to prevent environmental contaminants from entering the enclosure.

The enclosure 38 also includes a blower housing 46 that contains a fan or blower assembly. In operation, air enters the system 16 though the air intake 20 and is directed to the flexible conduit 22 by the fan assembly within the housing 46. The air intake 20 is disposed along the entire length of the enclosure 38 to provide a large air intake surface area. The large surface area may allow a large amount of air to enter the system 16 and also may provide an increased surface area for filtration.

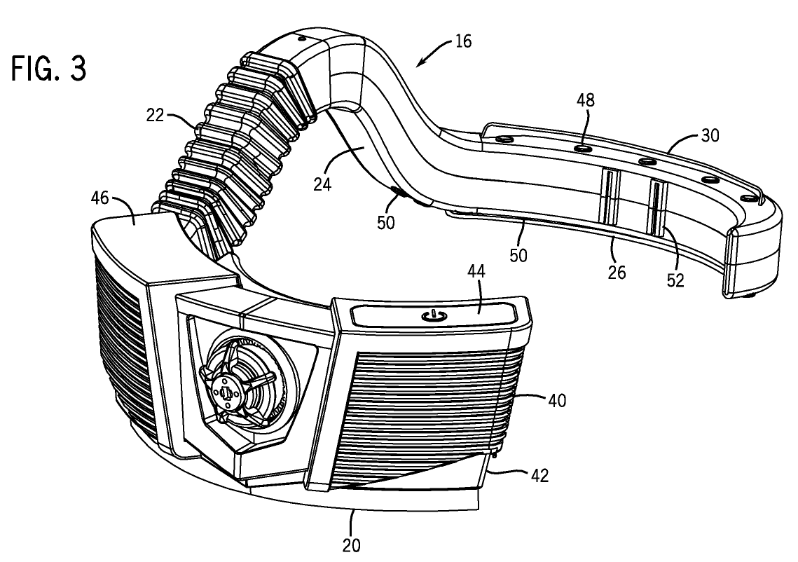

FIG. 3 illustrates the airflow system 16 detached from the headgear. As noted above, the airflow system 16 may be permanently integrated into the headgear or may be an auxiliary component that can be attached to the headgear. Air may enter through the air intake 20, flow through the flexible conduit 22 into the manifold 24, and exit the airflow system through the upper and lower vents 30 and 26. The upper and lower vents 30 and 26 each include tubes 48 and 50, respectively, for directing air from the vents. Specifically, the tubes 48 extend within the top of the manifold 24 to direct air in a transverse, upward direction out of the manifold. The vent 30 is located above the tubes 48 and directs the transverse air exiting the tubes 48 laterally towards the rear of the airflow system. In certain embodiments, each upper tube may have its own vent. The lower vent 26 includes the tubes 50 that extend within the bottom of the manifold to direct air transversely downward across a user's face. The lower vent 26 may be adjustable to change the angle of the air exiting the tubes 50.

The inner surface of the manifold 24 includes mounting brackets 52 that may be used to attach the airflow system 16 to the headgear. The mounting brackets 52 may be connected to tabs on the headband, such as tabs for attaching a sweat band. The mounting brackets 52 also may be attached to an adapter bracket that may be mounted to the headgear. In certain embodiments, the inner surface of the manifold 24 also may include one or more tubes extending from the inner surface of the manifold to direct air laterally toward the forehead of a user. The lateral tubes may function to provide cooling to a user's forehead by evaporating sweat that may collect on a user's forehead or sweat band.

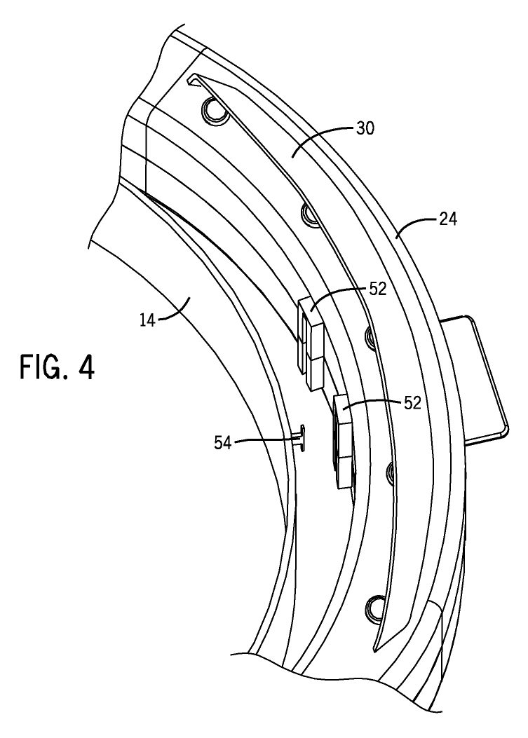

FIG. 4 illustrates attachment of the mounting brackets 52 to the headgear 14. The headgear 14 includes a tab 54, which in certain embodiments may be T-shaped, that extends outwardly from the headgear 14. The tab 54 may be inserted or slid into the mounting bracket 52 to affix the manifold 24 to the headgear 14. In other embodiments, the mounting brackets may be replaced by sockets for receiving the tabs. Of course, the number of mounting brackets 52 and tabs 54 may vary. In certain embodiments, the manifold 24 may be constructed of a flexible material to allow the manifold 24 to flex with the headgear 14 as the headgear conforms to a user's head.

In other embodiments, the manifold 24 may be constructed of a rigid material. In these embodiments, an adapter bracket may be used to attach the headgear 14 to the manifold 24. The adapter bracket may provide additional flexibility and allow the headgear 14 to flex and conform to a user's head. The adapter bracket also may be used to attach the manifold 24 to existing headgear that does not employ tabs suitable for attachment to the brackets 52.

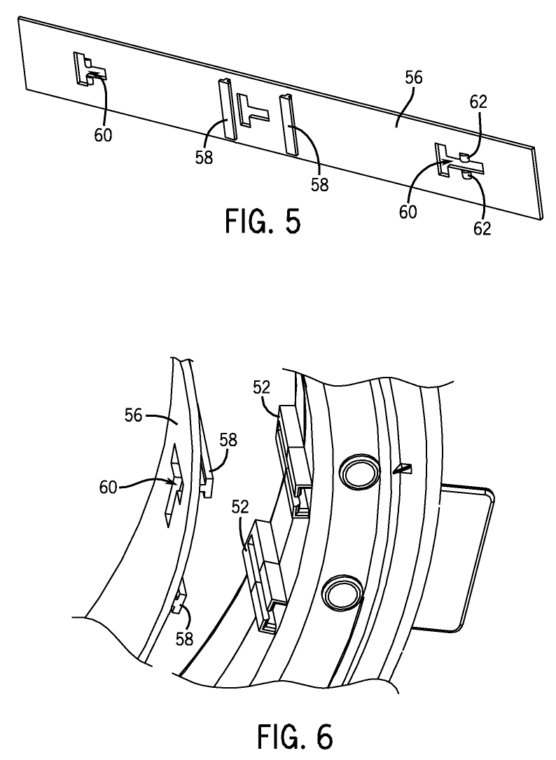

FIG. 5 illustrates an exemplary bracket 56 that may be used to attach the manifold to the headgear. The bracket 56 may be constructed of plastic or other suitable material. The bracket 56 includes rails 58 that extend outward from the surface of the bracket. The rails 58 may be molded into the bracket or affixed to the bracket surface and may be used to attach the bracket 56 to the manifold 24. In certain embodiments, the rails 58 may have a T-shaped profile that fits into the mounting brackets 52, shown in FIG. 4.

The bracket 56 also includes apertures 60 for attaching the headgear to the bracket 56. As shown, the apertures 60 include a generally T-shaped cross-section designed to accommodate a T-shaped tab of the headgear, such as the tab 54 shown in FIG. 4. Of course, in other embodiments, the apertures may be of different shapes and sizes designed to accommodate other types of tabs, brackets, or fixtures located on the headgear. Ridges 62 located near the apertures 60 may secure the headgear to the bracket. For example, tabs of the headgear may be inserted into the apertures 60 and then adjusted horizontally to slide over the ridges 62 and snap into place. Of course, other types of tabs brackets or mechanical fasteners, such as clips, may be used to hold the headgear in place.

FIG. 6 is a detailed view of the attachment that may occur between the bracket 56 and the manifold 24. The bracket 56 may be slid upward into the mounting bracket 52. The rails 58 located on the bracket 56 may be slid into the mounting brackets 52. The rails 58 and brackets 52 function to secure the rigid manifold to the sweatband mounts on the headgear while also allowing the headgear to flex and conform to the user's head.

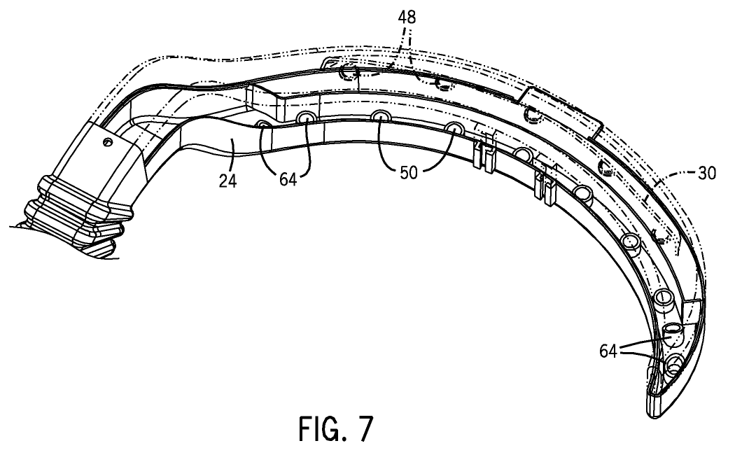

FIG. 7 illustrates the interior of the manifold 24. The upper vent 30 directs air exiting the top of the manifold 24. The air exiting the top of the manifold 24 is directed upward by upper tubes 48 and then directed over the back of the user's head by the vent 30. The air also may exit the bottom of the manifold 24 through the tubes 50. The tubes 50 may direct the air in a downward direction across and onto a user's face. The bottom of the manifold 24 also includes side tubes 64. These tubes may be angled, for example at thirty degree and sixty degree angles, to direct air toward the sides of a user's face. By laterally projecting the air toward the rear of the airflow system, the tubes may provide positive pressure around the user's face to prevent unfiltered air from entering the user's breathing zone. The tubes may be disposed mostly within the interior of the manifold 24 with only a small portion extending outside of the surface of manifold 24. In certain embodiments, the tubes may be one quarter inches in height or less.

The tubes 48 and 50 are intended to direct air in a transverse direction as it exits the manifold 24. Of course, the number, length, diameter, and angle of top tubes, bottom tubes, and side tubes may vary depending on the air flow requirements, manifold configurations, and other design factors. Further, in certain embodiments, the tubes may be adjustable to direct air in various directions. For example, the bottom tubes 50 may be rotatable to angle air across a user's face. In certain embodiments, the tubes also may be rotatable to decrease or increase the amount of air flow through the tubes. For example, a user may close certain bottom tubes to allow increased air flow through the remaining open bottom tubes. In another example, the top tubes may be closed when the welding helmet is raised, allowing increased air flow through the bottom tubes. In yet other embodiments, the tubes 48 and 50 may be replaced by internal chambers within the manifold 24. In these embodiments, holes may then be stamped or punched in top and bottom manifold surfaces to allow air to exit from the internal chambers. The internal chambers may be positioned to promote airflow in a transverse direction.

In other embodiments, tubes may be positioned to direct air laterally from the manifold 24. For example, tubes may extend from the interior wall of the manifold to direct air toward a user's forehead. In certain embodiments, the tubes 48 and 50 may be replaced by tubes extending from the exterior wall of the manifold to direct air laterally outward toward the welding helmet 10 (FIG. 1). In these embodiments, the welding helmet may include a deflector structure for directing the air exiting the manifold transversely toward a user's face and breathing zone. The deflector structure also may direct some of the air up and over a user's head generally toward the rear of the airflow system.

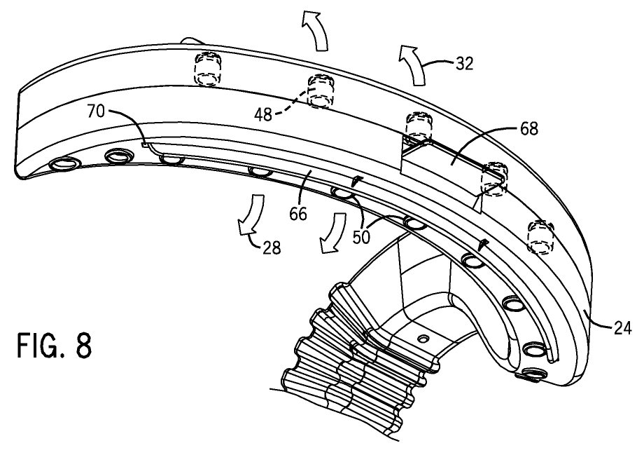

FIG. 8 is a bottom perspective view of the manifold 24. As noted above with respect to FIG. 7, the upper tubes 48 direct air transversely upward and rearward, as generally indicated by arrows 32, to maintain a positive pressure. The bottom tubes 50 direct air transversely downward across a user's face to provide filtered air to the user's breathing zone. A deflector 66 may slide into and out of the manifold 24 to further direct the airflow. The deflector 66 includes a tab 68 that a user may move upwards or downwards to change the direction of the air 28 exiting the tubes 50. For example, a user may move the tab 68 upwards to place the deflector 66 completely or partially within the manifold 70 and allow air to flow past a user's face. In another example, a user may pull the tab 68 in a downward direction to increase the portion of the deflector that extends from the manifold 24 to direct air toward the user's forehead or mouth. The deflector 66 may be curved to follow the contour of the manifold 24. The manifold 24 includes a slot 70 for receiving the deflector 66. The slot (or deflector chamber) may be sealed off from the manifold to prevent air from exiting the manifold through the slot. The interior wall of the slot 70 may include ridges to hold the deflector 66 in position. In other embodiments, teeth, ratchets, or other mechanical means may be used to maintain the position of the deflector 66. Further, multiple deflectors may be included within the manifold and one or more of the deflectors may rotate in addition to, or instead of, sliding. The deflector may also be positioned to move externally to the manifold.

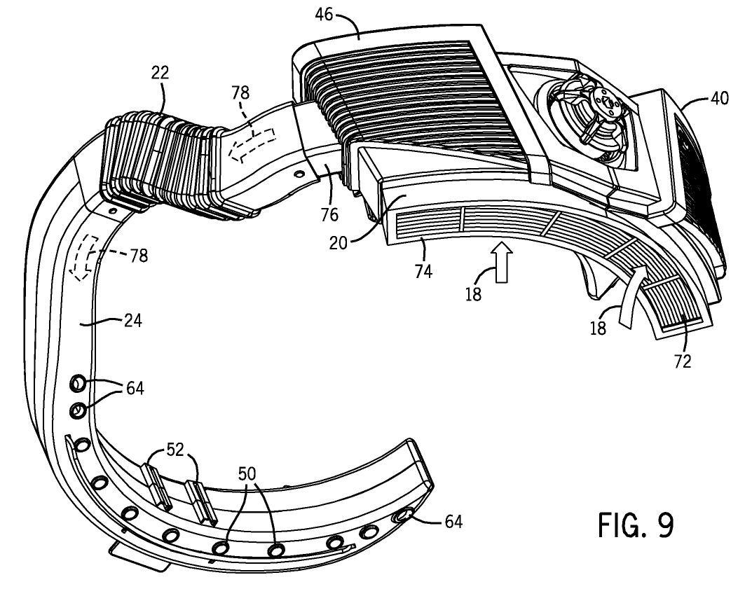

FIG. 9 is a bottom perspective view of the airflow system illustrating the air flow into and within the airflow system. The air intake 20 includes a filter 72 that may be held in place by a cover 74. The cover 74 may be removable from the air intake 20 so that the filter 72 may be replaced. However, in other embodiments, the cover 74 may be omitted and the filter 72 may be interference fit within the intake 20. The filter may be a dust filter, electrostatic air filter, carbon filter, HEPA filter, or other suitable type of filter. In certain embodiments, a white filter may be used so that a user can detect when a filter is dirty and requires changing. In other embodiments, the airflow system 16 may include a detection mechanism for determining when the filter should be replaced. For example, the detection mechanism may measure electrical impendence or detect reduced air flow and emit an audible signal, light, or other notification, to alert the user to replace the filter or to insert a filter if one is not present. In certain embodiments, the airflow system may be configured to remain off until the filter is replaced. In certain embodiments, sensors within the airflow system might detect the presence of a filter, and prevent use of the system if the filter is absent. As shown, one large filter fits within the air intake 20 that extends along the entire base of the enclosure 38 to provide a large surface area for filtration. However, in other embodiments, the filter may be disposed over only a portion of the enclosure 38, or multiple filters may be used. Of course, in other embodiments, the filter may be located in other areas of the airflow system, such as within the flexible tube or within the manifold.

Air may enter the airflow system through the air intake 20, as generally indicated by the arrows 18. The air intake 20 is disposed on the rear of the airflow system so that the air entering the airflow system is located on the other side of the user from the welding area. As air enters the intake 20, the air 18 may pass through the filter 72 to remove particles and/or gases from the air. A fan within the enclosure 46 may draw the air from the air intake 20 and direct the air radially through the flexible conduit 22. The flexible conduit may be connected to the enclosure 38 by a tube 76 extending from the enclosure 38. The tube 76 may be a rigid tube constructed of plastic or other suitable material and integrally molded into the enclosure 38. The tube 76 may have internal ribs and a seal or gasket for connecting to the flexible conduit 22. The flexible conduit may be removable to allow the user to clean the manifold and tubing.

Within the flexible conduit 22, the air may flow as generally indicated by the dashed arrows 78 to the manifold 24. The flexible conduit 22 generally may be constructed of material that is softer than the headgear. For example, the flexible conduit may be constructed of linear polyethylene combined with a copolymer. The flexible conduit may be designed to stretch and shrink as the size of the headgear is adjusted. Thus, the size of the air flow path also may expand or contract. In other embodiments, the flexible conduit 22 may be constructed of a flexible plastic or other material that does not compress or shrink with the circumferential adjustment of the headgear, but instead flexes by bending or bowing outward. In certain embodiments, the air 78 may be cooled as it flows through the flexible conduit 22. For example, the tubing 22 may include a removable cooling source, such as an insertable gel pack or ice pack. Such a cooling source might be inserted near the air intake, air exit, or elsewhere within the path of airflow. The cooling source may provide thermoelectric or evaporative cooling. The air 78 may flow from the flexible conduit 22 to the manifold 24 and exit the manifold through the top and bottom tubes 48 and 50, as described with respect to FIG. 8. In general, the path of the airflow from the intake 20 to the manifold exits may be sealed to prevent air leakage or the entraining of unfiltered air.

In other embodiments, the flexible conduit may include a pair of rigid tubes configured to slide to allow the conduit to stretch and shrink. For example, a tube of a smaller diameter may be configured to slide within a larger diameter tube to increase or decrease the length of the flexible conduit. The inner and outer tubes, although individually fixed in length, may function together to create an adjustable and flexible conduit. In these embodiments, a rubber gasket may provide a seal between the two tubes.

The flexible conduit 22 also may be replaced by a conduit of a fixed length that does not adjust with the circumferential adjustment of the headgear. In these embodiments, the head gear adjustment knob may be located on the side of the headgear opposite from the fixed length conduit. The side adjustment knob may adjust the overall circumferential size of the headgear by increasing or decreasing the length of the portion of the headgear strap that is located on the same side of the headgear as the adjustment knob.

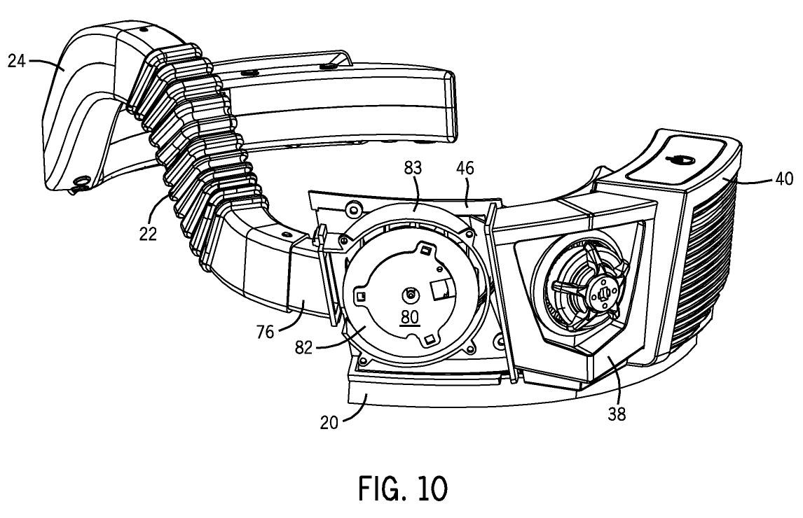

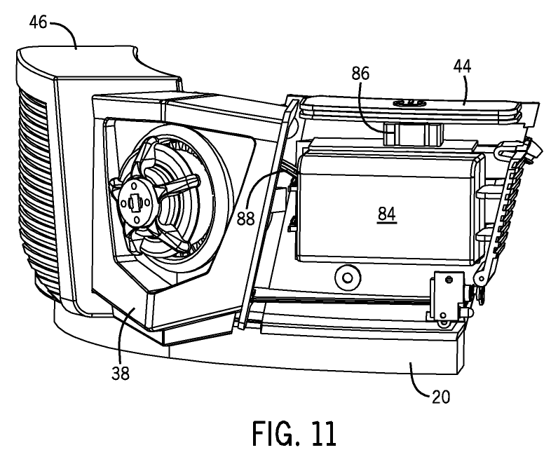

FIG. 10 illustrates the airflow system with a portion of the fan enclosure 46 removed to reveal the internal components. The fan assembly includes a motor 80 and a fan or blower 82 disposed within the enclosure 46. A baffle 83 may surround the fan and provide apertures for attaching the fan assembly to the fan enclosure 46. The baffle 83 also may function to direct air radially into the flexible tubing 22. The fan 82 may include a centrifugal fan or other suitable fan powered by an AC or DC direct or variable drive motor.

FIG. 11 illustrates the airflow system 16 with a portion of the power supply enclosure 44 removed to reveal the internal components. The enclosure 44 includes a battery 84 and a circuit 86 for powering the airflow system. In certain embodiments, the circuit 86 may include one or more integrated circuits. Further, the circuit may be coated with a conformal coating to prevent metallic dust from attaching to or contacting the components of the circuit. Wires 88 may connect battery terminals to the motor for powering the fan. In certain embodiments, the wires 88 may be routed along the top portion of the enclosure 38. The battery may be a lithium polymer rechargeable battery, lithium-ion battery, or other suitable type of battery. In certain embodiments, the battery may be removable from the enclosure 44 so that is may be charged by an external charging station. According to exemplary embodiments, the battery may have a voltage of 8.4 volts when fully charged.

The circuit 86 may be located between the on/off switch 44 and the battery 84 such that the pressing of the switch 44 engages the circuit and battery to power the airflow system. However, in other embodiments, the circuit 86 may be located along a side of or below the battery 84 and connected to the switch 44 by wires or other electrical or mechanical components. In certain embodiments, the switch 44 may be configured to detect when the welding helmet is positioned down over a user's face. In these embodiments, the switch may apply power to the unit only when the helmet is in the down position. Further, a delay timer may be included to purge any residual fumes after the helmet is raised. However, in other embodiments, the switch may continue to power the airflow system when the helmet is in the raised or lowered position.

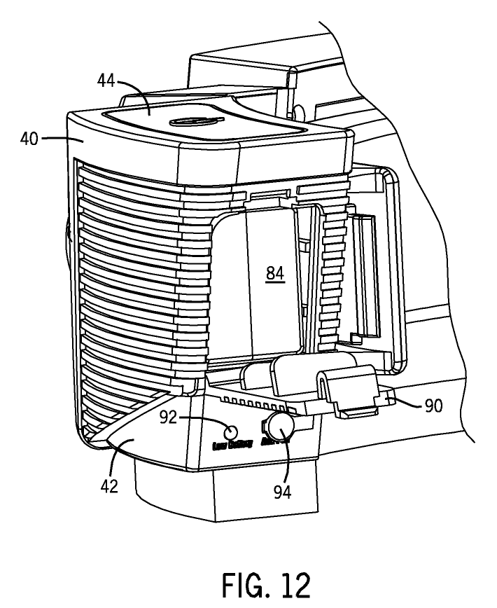

FIG. 12 is a side perspective view of the power supply enclosure 40. A cover 90 may be removed to access the battery 84. The cover may be hinged to the enclosure 40 and may swing up, down, or out sideways from the enclosure 40. The cover also may snap or slide in place or be attached to the battery. In other embodiments, the cover may be completely removable from the enclosure 40 and/or attached to the enclosure 40 using a strap or other suitable attachment method. In other embodiments, the battery may be completely integrated into the unit so that is not removable. The enclosure 40 also includes a low battery indicator 92. The low battery indicator 92 may be a light emitting diode or other type of visual or audible indicator that notifies a user when the battery requires replacement or charging. In certain embodiments, the low battery indicator 92 may change colors or intensities to indicate the battery level. For example, the battery indicator may blink when the battery is approaching a low level and may remain lit once the battery level falls below a threshold.

The enclosure 40 also includes a port 94 for receiving auxiliary power. The port 94 may be configured to receive an AC or DC power source and may be used to provide additional power to the unit to prolong the operating time. In certain embodiments, the port 94 may be used to receive power from an additional battery that may be worn on the user's belt. In other embodiments, the port 94 may be used to plug the unit into a power receptacle or power supply integrated into another piece of equipment, such as a welding torch. In certain embodiments, the airflow system may receive power solely through the port 94 so that the battery 84 may be removed to reduce the weight of the airflow system.

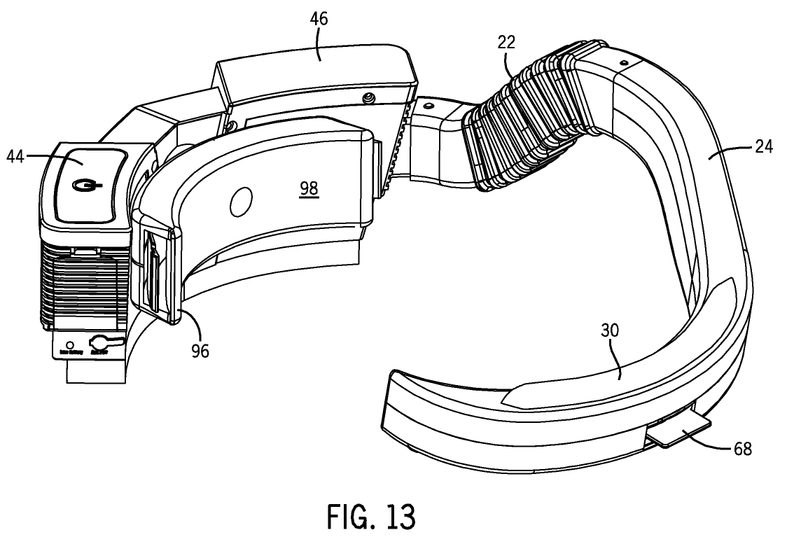

FIG. 13 illustrates a switch 96 that may be included within the airflow system 16. The switch 96 may engage when the airflow system is worn by a user and disengage when the airflow system is removed from a user's head. For example, the switch 96 may be a pressure sensitive switch located on the interior side of the enclosure 38 to rest against the back of the user's head when the airflow system is worn. However, in other embodiments, the switch may include an electrical switch, optical switch, or other type of mechanical switch. The switch 96 may be disposed on a cushion pad 98 that provides comfort and distributes weight on a user's head.

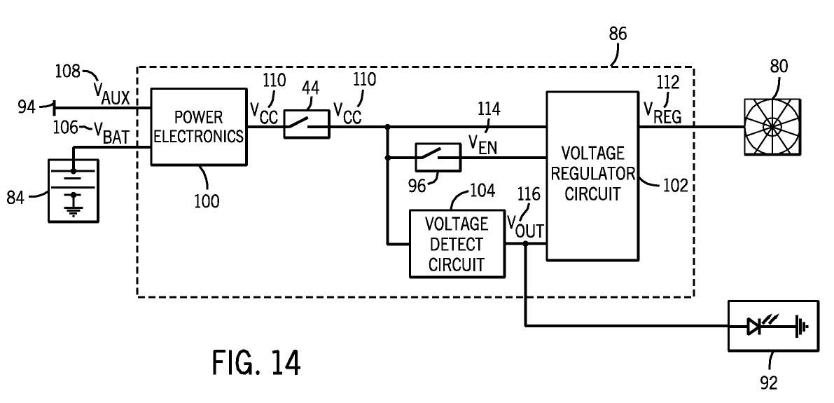

FIG. 14 illustrates an exemplary circuit that may be used to power the airflow system. The circuit 86 includes a power electronics circuit 100, a voltage regulator circuit 102, and a voltage detect circuit 104. The circuit 86 may be configured to receive power through the auxiliary power port 94, from the battery 84, or from a combination thereof. The potential energy of the battery may be represented by V_{BAT} 106, which in certain embodiments may have a value on 8.4 volts when the battery 84 is fully charged and a value of approximately 7.4 volts during normal operation. The electric potential of the auxiliary power source may be represented by the V_{AUX} 108. The power electronics circuit 100 may include a series of diodes for combining V_{BAT} 106 and V_{AUX} 108 into a positive voltage supply V_{CC} 110. In certain embodiments, the power electronics circuit 100 may include a charge circuit for charging the battery 84 through the auxiliary power port 94.

When engaged, the on/off switch 44 may allow current to flow from the charge circuit to the fan 80. In certain embodiments, both the on/off switch 44 and the auto switch 96 must be engaged to power the fan 80. When engaged, the auto switch 96 may provide an enable signal represented by the V_{EN} 114 to the voltage regulator circuit 102. In certain embodiments, the voltage regulator circuit 102 may include a pulse width modulation step-down DC/DC converter electrically coupled to diodes, capacitors, inductors, and resistors to provide a constant voltage to fan 80. The constant voltage may be generally represented by the V_{REG} 112, and in certain embodiments, may be 5 volts. The voltage regulator may be a switching or linear regulator. The voltage regulator circuit 102 may further include a potentiometer to allow adjustment of the air flow through the fan 80. For example, the potentiometer may be controlled by an adjustable knob located on the airflow system and may change the voltage supplied to the fan 80. In other embodiments, the potentiometer may adjust the duty cycle of constant voltage pulses applied to the fan 80.

The circuit 86 also includes a voltage detect circuit 104 that monitors the battery voltage and shuts off the airflow system if the battery voltage falls below a specified threshold to prevent permanent damage to the battery. In certain embodiments, the voltage detect circuit 104 may include a micropower, latching voltage monitor coupled to capacitors and resisters to specify the threshold voltage value. In certain embodiments, the threshold value may be 5.8 volts and the latch may be reset by toggling the on/off switch or by removing and replacing the battery. In other embodiments, the voltage detect circuit 104 may include resistors, transistors and diodes electrically coupled to disengage the fan 80 when the voltage falls below the specified threshold level. The output of the voltage detect circuit may be generally represented by V_{OUT}. When the value of the V_{OUT} is pulled low, the voltage regulator circuit 102 may disengage the fan 80 and the voltage detect circuit 104 may engage the low battery indicator 92. As may be appreciated, many additional components such as resistors, capacitors, inductors, diodes, and transistors, may be included within the circuit 86.

The airflow systems described above are intended to provide an integrated airflow system that may be included within a welding helmet. The airflow system is designed to provide air flow to the user's face and breathing zone and may further provide positive pressure to prevent unfiltered air from entering the breathing zone. The airflow system is designed to be portable, light weight, and compatible with existing welding helmets.

While only certain features of the invention have been illustrated and described herein, many modifications and changes will occur to those skilled in the art. For example, the flexible conduit and manifold may wrap around the left or right side of a user's head or traverse over the top of the user's head. Further, the location and number of components such as the motor, fan, filter, and battery may vary. In another example, the filter may be disposed to receive air horizontally, or a length of tubing may be attached to the air intake to provide an air source from a farther distance away. In yet another example, fabric may be used to seal the back of a user's head or neck to the helmet shell. The fabric may impede the entrainment of unfiltered air into the air flow system and may be used in addition to or instead of the tubes providing positive pressure. It is, therefore, to be understood that the appended claims are intended to cover all such modifications and changes as fall within the true spirit of the invention.

1. An airflow system for a welding helmet, the airflow system comprising:
a headgear configured to adjustably fit the head of a wearer
a rear portion secured to the headgear and configured to receive air;
a front portion configured to direct the air toward the face of the wearer; and
a flexible conduit that fluidly connects the front and rear portions and allows a circumferential adjustment of the headgear.

2. The system of claim 1, wherein the front portion comprises an air directing structure configured to project the air towards the wearer's face, towards the top of the wearer's head, and towards the sides of the wearer's head to create an outward flow.

3. The system of claim 2, wherein the outward flow reduces entrainment of unfiltered air.

4. The system of claim 1, comprising a fan or a blower configured to draw the air into the rear portion and direct the air through the flexible conduit towards the front portion.

5. The system of claim 4, comprising a power source for powering the fan or the blower.

6. The system of claim 5, wherein the power source comprises a rechargeable battery mounted in the rear portion.

7. The system of claim 5, comprising a circuit configured to provide a regulated voltage from the power source to the fan or the blower and to disable the fan or the blower when a voltage of the power source falls below a threshold.

8. The system of claim 5, wherein the power source comprises a port for receiving an external power supply.

9. The system of claim 5, wherein the port is configured to charge a battery of the rear portion when the port is connected to a power supply.

10. The system of claim 1, comprising an air intake mounted in the rear portion and configured to receive a filter and to direct the air through the filter.

11. The system of claim 1, wherein the front portion includes transverse tubes disposed in opposite surfaces of a manifold for transversely directing the air to create a transverse flow.

12. The system of claim 11, wherein the manifold includes angled tubes configured to laterally project the air towards the rear portion to reduce entrainment of unfiltered air into the transverse flow.

13. The system of claim 11, wherein the manifold includes a deflector configured to direct the transverse flow.

14. The system of claim 1, comprising a mounting structure configured to integrate the headgear into the welding helmet.

15. The system of claim 1, comprising a fabric structure configured to be disposed between the welding helmet and the wearer to reduce entrainment of unfiltered air.

16. An airflow system for a welding helmet, the airflow system comprising:
a mounting structure configured to integrate the airflow system into welding helmet headgear;
an air intake portion configured to receive air near the rear of the headgear;
an air ventilation portion configured to direct air transversely near the front of the headgear; and
a flexible conduit configured to follow the curvature of the headgear, to allow circumferential adjustment of the headgear, and to direct air from the air intake portion to the air ventilation portion.

17. The system of claim 16, wherein the air intake portion comprises:
a filter configured to filter the air;
a fan and motor assembly for drawing the air through the filter and directing the air to the air ventilation portion;
a rechargeable battery configured to provide power to the fan and motor assembly;
a circuit configured to regulate the power; and
a user accessible adjustment knob configured to adjust the flexible conduit and circumferential size of the headgear.

18. The system of claim 16, wherein the air ventilation portion comprises:
a plurality of jets configured to direct air from the system in a transverse flow; and
an air directing structure configured to laterally project a portion of the transverse flow towards the air intake portion to create an outward flow.

19. The system of claim 16, wherein the mounting structure comprises an adapter bracket configured to attach to the headgear and to the air flow system.

20. The system of claim 16, comprising a switch configured to enable the system when the headgear is worn by a user.

21. The system of claim 16, comprising a manually controlled switch configured to enable and disable the system.

22. A method for making an adjustable headgear for a welding helmet, the method comprising:
coupling a first end of a flexible tubing to an outlet of a fan assembly configured to receive air through an intake and direct the air towards the outlet;
coupling a second end of the flexible tubing to an inlet of a manifold configured to receive a lateral airflow and expel the air from the manifold in a transverse airflow; and
attaching the fan assembly and the manifold to a strap configured to support a welding helmet such that the flexible tubing expands or contracts in response to a circumferential adjustment of the strap.

23. An airflow system for a welding helmet, the system comprising:
headgear configured to adjustably fit the head of a wearer;
a rear portion secured to the headgear and configured to receive air;
a front portion configured to direct the air toward the face of the wearer and to direct the air toward the rear portion, to create a positive pressure that reduces entrainment of unfiltered air.

24. The system of claim 23, wherein the front portion comprises transverse tubes disposed in opposite surfaces of a manifold for transversely directing the air.

25. The system of claim 24, wherein the front portion comprises an air directing structure configured to laterally project air exiting the tubes toward the rear portion.
